(19) United States
(12) Patent Application Publication
(10) Pub. No.: US 2022/0296145 A1
(43) Pub. Date: Sep. 22, 2022

(54) COATING ELECTRODES OF MEDICAL DEVICES

(71) Applicant: Nir Geva, Ness Ziona (IL)

(72) Inventor: Nir Geva, Ness Ziona (IL)

(21) Appl. No.: 17/633,952

(22) PCT Filed: Aug. 6, 2020
(86) PCT No.: PCT/IB2020/057422

§ 371 Date: Feb. 8, 2022

Related U.S. Application Data
(60) Provisional application No. 62/883,668, filed on Aug. 7, 2019.

(51) Int. Cl. A61B 5/263; A61B 5/00

(52) U.S. Cl. CPC A61B 5/263; A61B 5/6898

(57) ABSTRACT

A method for coating an electrode of a medical device, the method consists essentially of coating the electrode of the medical device with chromium monosilicide.

### CROSS REFERENCE

This application claims priority of U.S. provisional patent Ser. No. 62/883,668 filing date Aug. 7, 2019.

### BACKGROUND OF THE INVENTION

Health monitoring capabilities are included in user devices such as smartphones, smart watches, media players and wearable devices.

Various measurements require a monitored person to contact one or more electrodes.

These electrodes should be coated in order to increase their durability and for esthetic reasons.

Wet electrodes may be temporarily coated with gel—but such a solution is not feasible in various situations. The gel has a limited lifespan, and may be problematic to carry.

Monitoring physiological signals for organs such as finger requires using multilayer dry electrodes.

Multilayer dry electrodes are coated with multiple coating layers of different coating materials. For example a plastic electrode may be coated with multiple layers of different materials such as a first layer of copper, a second layer of nickel and a third layer that includes at least one material out of silver, silver-chloride, rhodium, palladium or gold. A metal electrode is also coated with multiple layers of different materials.

U.S. Pat. No. 10,443,143 illustrates a method for coating with multiple layers that include a trivalent chromium that is formed over an intermediate layer and over a base layer of nickel phosphorous. The coated object is subjected to one or more heat treatments to harden the coating and to produce multiphase layers including at least one layer containing crystalline Ni and crystalline Ni3P and at least one layer containing crystalline Cr. The intermediate layer can consist of copper, molybdenum, a metal alloy or a non- metallic solid, such as an oxide, nitride or carbide of a metal.

The multiple layers may introduce noises and distortions introduced to a signal (from the exterior of the coating to the electrode itself). Noises may also be generated due to difference between the impedance of a finger and an impedance of a coated electrode. In order to minimize the noise, the impedance of the finger and the impedance of the external layer of the electrode should be as close as possible.

There is a growing need to provide efficient coatings for such electrodes.

### SUMMARY

There may be provided a method for coating an electrode of a medical device, the method may consist essentially of coating the electrode of the medical device with chromium monosilicide.

The coating may consist essentially of forming a single layer of chromium monosilicide.

The coating may consist of forming a single layer of chromium monosilicide.

The coating may consist essentially of forming multiple layers of chromium monosilicide.

The coating may consist of forming multiple layer of chromium monosilicide.

The coating may be preceded by removing fats from the electrode.

The coating of the medical device with chromium monosilicide provides a coating with a predefined resistance.

The coating of the medical device with chromium monosilicide provides a coating with a predefined color.

There may be provided a medical device comprising an electrode, wherein the electrode may be coated by a coating that consist essentially of chromium monosilicide.

The coating may consist essentially of a single layer of chromium monosilicide.

The coating may consist of a single layer of chromium monosilicide.

The coating may consist essentially of multiple layers of chromium monosilicide.

The coating may consist of multiple layer of chromium monosilicide.

The electrode may be a fat removed electrode.

The coating may be of a predefined resistance.

The coating may be of a predefined color.

The electrode may be made of at least one out of nirosta steel, nickel, brass.

The electrode may be made of at least one out of polymer or plastic.

The electrode may be made of at least one out of polycarbonate and thermoplastic polymers.

A coated electrode that may consist essentially of an electrode and a coating that may consist essentially of chromium monosilicide.

The coating may consist essentially of a single layer of chromium monosilicide.

The coating may consist of a single layer of chromium monosilicide.

The coating may consist essentially of multiple layers of chromium monosilicide.

The coating may consist of multiple layer of chromium monosilicide.

The electrode may be a fat removed electrode.

The coating may be of a predefined resistance.

The coating may be of a predefined color.

The electrode may be made of at least one out of nirosta steel, nickel, brass.

The electrode may be made of at least one out of polymer or plastic.

The electrode may be made of at least one out of polycarbonate and thermoplastic polymers.

There may be provided a method for measuring at least one physiological signal, the method may include performing a measurement process that comprises obtaining at least one physiological signal using a coated electrode, the coated electrode that may consist essentially of an electrode and a coating that may consist essentially of chromium monosilicide.

The coating may consist essentially of a single layer of chromium monosilicide.

The coating may consist of a single layer of chromium monosilicide.

The coating may consist essentially of multiple layers of chromium monosilicide.

The coating may consist of multiple layer of chromium monosilicide.

The electrode may be a fat removed electrode.

The coating may be of a predefined resistance.

The coating may be of a predefined color.

The electrode may be made of at least one out of nirosta steel, nickel, brass.

The electrode may be made of at least one out of polymer or plastic.

The electrode may be made of at least one out of polycarbonate and thermoplastic polymers

There may be provided a method for evaluating a person, the method may include obtaining at least one physiological signal using a coated electrode, the coated electrode that may consist essentially of an electrode and a coating that may consist essentially of chromium monosilicide; and processing the at least one physiological signal to provide an evaluation regarding the person based on the at least one physiological signal.

The coating may consist essentially of a single layer of chromium monosilicide.

The coating may consist of a single layer of chromium monosilicide.

The coating may consist essentially of multiple layers of chromium monosilicide.

The coating may consist of multiple layer of chromium monosilicide.

The electrode may be a fat removed electrode.

The coating may be of a predefined resistance.

The coating may be of a predefined color.

The electrode may be made of at least one out of nirosta steel, nickel, brass.

The electrode may be made of at least one out of polymer or plastic.

The electrode may be made of at least one out of polycarbonate and thermoplastic polymers.

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a medical device should be applied mutatis mutandis to a method executed by the medical device.

Any reference in the specification to a medical device should be applied mutatis mutandis to a method of manufacturing the medical device.

Any reference in the specification to a coated electrode should be applied mutatis mutandis to a method executed by the coated electrode .

Any reference in the specification to a coated electrode should be applied mutatis mutandis to a method of manufacturing the coated electrode .

Any reference in the specification to a method should be applied mutatis mutandis to a medical device configured to execute the method and/or to a medical device manufactured using, at least in part, the method. Non-limiting examples of medical devices are illustrated in U.S. patent application Ser. No. 16/325,391 JACKET FOR MEDICAL DEVICE, in U.S. patent application Ser. No. 16/586,934 METHOD, DEVICE AND SYSTEM FOR NON-INVASIVELY MONITORING PHYSIOLOGICAL PARAMETERS, in U.S. Pat. No. 10,251,603 titled SYSTEMS AND METHODS FOR VITAL SIGNS MONITORING WITH EAR PIECE, in U.S. patent application Ser. No. 16/650,010 METHOD AND SYSTEM FOR OBTAINING PHYSICAL CONDITION THAT LEAD TO A DEFIBRILLATOR CONUTERSHOCK, in U.S. patent application Ser. No. 16/762,581 titled HEALTH MONITORING DEVICE THAT INCLUDES A COMPACT OXIMETER, all being incorporated herein by reference.

Any reference in the specification to a method should be applied mutatis mutandis to a coated electrode configured to execute the method and/or to a coated electrode manufactured using, at least in part, the method.

Any combination of any module or unit listed in any of the figures, any part of the specification and/or any claims may be provided.

Any combination of any steps of any method illustrated in the specification and/or drawings may be provided.

Any combination of any subject matter of any of claims may be provided.

The term “consisting essentially of” is used to limits the scope of a method or a medical device of a coated electrode to the specified materials or steps “and those that do not materially affect the basic and novel characteristic(s)” of the invention.

The term “consisting” excludes any element, step, or ingredient of a medical device or a coated electrode not explicitly specified.

It has been found that coating electrodes with chromium monosilicide provides unexpected benefits—some illustrated below.

The chromium monosilicide may be used to coat electrodes made of various materials such as metals including but not limited to nirosta steel, nickel, brass, as well as polymer or plastic electrodes (such as polycarbonate (PC), thermoplastic polymers such as Acrylonitrile butadiene styrene(ABS), PC-ABS, and the like.

The electrode may be coated only with chromium monosilicide. After forming the shape of the electrode, it will go through a cleaning process to remove any greasy material or any microns elements from the surface of the electrode to insure maximum adhesion of the chromium monosilicide during the coating process.

The beauty and a benefit resulting from this type of coating, compare to standard coating of bio-impedance electrodes used today in the industry, is that it doesn't require any preparation prior to the coating of the top layer. For example, standard dry electrodes, require multi-layers of coating, starting with cooper, then nickel and finally, silver or palladium, rhodium, etc. before moving from one step of coating to the other, the electrode needs to be washed to remove residuals. This type of coating is clean, fast and doesn't require anything but pre-cleaning of the electrode before coating.

The coating process may include (a) cleaning the electrode from fats (for example cleaning the electrode with a degreaser), (b) performing non-conductive vacuum metallization or any other process in order to coat the cleaned electrode with the chromium monosilicide. The chromium monosilicide may be provided at different formats/states such as powder, bulks, solution, and the like.

An example for forming chromium monosilicide may include using a pack cementation process (see Stathokostopoulos, D; Chaliampalias, D; Tarani, E; Theodorakakos, A; Giannoulatou, V; Polymeris, G. S; Pavlidou, E; Chrissafis, K; Hatzikraniotis, E; Paraskevopoulos, K. M; Vourlias, G (2014). “Formation of the Thermoelectric Candidate Chromium Silicide by Use of a Pack-Cementation Process”. Journal of Electronic Materials. 43 (10): 3733-3739).

Another example for forming chromium monosilicide may include Vertical Gradient Freeze (see: A. Moll, S. Laborde, F. Barou, M. Beaudhuin “Centimetric CrSi₂ crystal grown by the vertical gradient Freeze method” Journal of Crystal Growth 534, 125505, May 2020).

The chromium monosilicide may be deposited on Copper using the process illustrated in “A. Liang, Q. Liu, B. Zhang and L. Ni “Preparation of Crystalline Chromium Coating on Cu Substrate Directly by DC Electrodepositing from Wholly Environmentally Acceptable Cr(III) Electrolyte” Materials Letters 119:131-134 March 2014.

The chromium monosilicide may be deposited using, for example electroplating or electroless plating.

The NCVM process, or as it is also called Physical vapor deposition (PVD) is a widely used technique in semiconductor integrated circuit (IC) manufacturing. Sputter deposition is a physical vapor deposition (PVD) method of depositing thin films by sputtering, that is ejecting, material from a “target,” that is source, which then deposits onto a “substrate,” such as a silicon wafer. Resputtering is re-emission of the deposited material during the deposition process by ion or atom bombardment. Sputtered atoms ejected from the target have a wide energy distribution, typically up to tens of eV (100,000 K). The sputtered ions (typically only about 1% of the ejected particles is ionized) can ballistically fly from the target in straight lines and impact energetically on the substrates or vacuum chamber causing resputtering. At higher gas pressures, they collide with the gas atoms that act as a moderator and move diffusively, reaching the substrates or vacuum chamber wall and condensing after undergoing a random walk. The entire range from high-energy ballistic impact to low-energy thermalized motion is accessible by changing the background gas pressure. The sputtering gas is often an inert gas such as argon. For efficient momentum transfer, the atomic weight of the sputtering gas should be close to the atomic weight of the target, so for sputtering light elements neon is preferable, while for heavy elements krypton or xenon are used. Reactive gases can also be used to sputter compounds. The compound can be formed on the target surface, in-flight or on the substrate depending on the process parameters. The availability of many parameters that control sputter deposition make it a complex process, but also allow experts a large degree of control over the growth and microstructure of the film. Sputtering is used extensively in the semiconductor industry to deposit thin films of various materials in integrated circuit processing. Thin antireflection coatings on glass for optical applications are also deposited by sputtering. Because of the low substrate temperatures used, sputtering is an ideal method to deposit contact metals for thin-film transistors. Perhaps the most familiar products of sputtering are low-emissivity coatings on glass, used in double-pane window assemblies. The coating is a multilayer containing silver and metal oxides such as zinc oxide, tin oxide, or titanium dioxide. Sputtering is also used to metalize plastics such as potato chip bags. A large industry has developed around tool bit coating using sputtered nitrides, such as titanium nitride, creating the familiar gold colored hard coat. (Reference: http://htelabs.com/virtual_fab/pvd_sputtering_deposition_physical_vapor_deposition_sputtering_thin_films_metals_alloys_dielectrics_dc_magnetron_rf_magnetron_sputter_deposition_reactive_sputtering_pvd.htm) The thickness of the chromium monosilicide coating determines the impedance of the coating—and thus a required impedance may be obtained by setting the thickness of the coating. The relationship between the impedance and the thickness may be calculated, estimated, simulated, tested in any manner. As a rule of thumb—the electrical resistivity of and the shape and size of the chromium monosilicide coating determine the impedance.

In 20 KHz frequency, the body impedance can varies from 1MOhm to 1.5MOhm, depending on skin dehydration, fat, greasy fingers and others.

As the thickness of the Cr—Si is so thin, we couldn't measure it and especially, the thickness variation between different coating time, we performed an ECG tests on live subjects to determine the best coating. In this analysis we concluded, that based on this body impedance range, the best coating is when we leave the electrodes in the coating chamber for 130 Minutes.

This coating (of 130 minutes) was compared to other electrodes coated with Cr—Si for less and more than 130 minutes, as well as to electrodes coated with Silver, Rhodium and stainless steel.

Based on this, we determine the manufacturing process to 130 Minutes of Cr—Si deposition as the optimum coating thickness.

The thickness also defines the color/shade of the coating. The Chromium silicide is a gray material. Once evaporated and coating the electrode, it start to form a gray color on the electrode, which goes and turn into black as long as the coating thickness increase. There is no additional pigment or supplementary color added to the process.

The coating process is simple, may result in a single coating layer, and may apply the chromium monosilicide directly on the electrode.

This coating provides an electrode that may be coated with a single layer of chromium monosilicide. In such an electrode there are not (or virtually no) noises.

It has been surprisingly found that an electrode coated with chromium monosilicide exhibits the following:
i. The noise introduced to signals that passed through the coating and reached the electrode was much smaller than the noise obtained with prior art electrodes.
ii. The attenuation of signals that passed through the coating and reached the electrode was much smaller than the attenuation obtained with prior art electrodes.
iii. The direct current level of the signals that passed through the coating and reached the electrode was more stable than the DC level of signals obtained with prior art electrodes.
iv. The electrode complied with various ISO 10993 tests.

The electrode may be used for any type of medical measurement or other purposes—for example oxygen saturation, ECG, blood pressure, and the like. The medical device may be any medical device that include electrodes—may be included in a mobile communication device such as a smartphone, may be included in a jacket or a cover that is in contact with a mobile communication device, may be a part of a dedicated medical device that differs from a smartphone—and the like.

There may be provided a method for measuring at least one physiological signal, such as ECG, EEG, EMG, GSR (galvanic skin response), the method may include performing a measurement process that comprises obtaining at least one physiological signal using a coated electrode, the coated electrode that consists essentially of an electrode and a coating that consists essentially of chromium monosilicide. The coated electrode may contact a person during the measurement.

There may be provided a method for evaluating a person, the method may include obtaining at least one physiological signal using a coated electrode, the coated electrode that consists essentially of an electrode and a coating that consists essentially of chromium monosilicide; and processing the at least one physiological signal to provide an evaluation regarding the person, for example, specific arrhythmia or specific brain activity (or lack of activity) based on the at least one physiological signal. The coated electrode may contact a person during the measurement.

While illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive.

Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

We claim:

1. A method for coating an electrode of a medical device, the method consists essentially of: coating the electrode of the medical device with chromium monosilicide.

2. The method according to claim 1 wherein the coating consists essentially of forming a single layer of chromium monosilicide.

3. The method according to claim 1 wherein the coating consists of forming a single layer of chromium monosilicide.

4. The method according to claim 1 wherein the coating consists essentially of forming multiple layers of chromium monosilicide.

5. The method according to claim 1 wherein the coating consists of forming multiple layer of chromium monosilicide.

6. The method according to claim 1 wherein the coating is preceded by removing fats from the electrode.

7. The method according to claim 1 wherein the coating of the medical device with chromium monosilicide provides a coating with a predefined resistance.

8. The method according to claim 1 wherein the coating of the medical device with chromium monosilicide provides a coating with a predefined color.

9. A medical device comprising an electrode, wherein the electrode is coated by a coating that consist essentially of chromium monosilicide.

10. The medical device according to claim 9 wherein the coating consists essentially of a single layer of chromium monosilicide.

11. The medical device according to claim 9 wherein the coating consists of a single layer of chromium monosilicide.

12. The medical device according to claim 9 wherein the coating consists essentially of multiple layers of chromium monosilicide.

13. The medical device according to claim 9 wherein the coating consists of multiple layer of chromium monosilicide.

14. The medical device according to claim 9 wherein the electrode is a fat removed electrode.

15. The medical device according to claim 9 wherein the coating is of a predefined resistance.

16. The medical device according to claim 9 wherein the coating is of a predefined color.

17. The medical device according to claim 9 wherein the electrode is made of at least one out of nirosta steel, nickel, brass.

18. The medical device according to claim 9 wherein the electrode is made of at least one out of polymer or plastic.

19. The medical device according to claim 9 wherein the electrode is made of at least one out of polycarbonate and thermoplastic polymers.

20. A coated electrode that consists essentially of an electrode and a coating that consists essentially of chromium monosilicide.

21. The coated electrode according to claim 20 wherein the coating consists essentially of a single layer of chromium monosilicide.

22. The coated electrode according to claim 20 wherein the coating consists of a single layer of chromium monosilicide.

23. A method for measuring at least one physiological signal, the method comprises performing a measurement process that comprises obtaining at least one physiological signal using a coated electrode, the coated electrode that consists essentially of an electrode and a coating that consists essentially of chromium monosilicide.

24. A method for evaluating a person, the method comprises obtaining at least one physiological signal using a coated electrode, the coated electrode that consists essentially of an electrode and a coating that consists essentially of chromium monosilicide; and processing the at least one physiological signal to provide an evaluation regarding the person based on the at least one physiological signal.
